(19) United States
(12) Patent Application Publication
(10) Pub. No.: US 2025/0160643 A1
(43) Pub. Date: May 22, 2025

(54) MEASUREMENT DEVICE, INFORMATION TERMINAL, AND OPERATION RECORDING MEDIUM OF INFORMATION TERMINAL

(71) Applicant: OMRON HEALTHCARE Co., Ltd., Kyoto (JP)

(72) Inventor: Tetsuzo OKAZAKI, Kyoto (JP)

(21) Appl. No.: 19/032,326

(22) Filed: Jan. 20, 2025

Related U.S. Application Data
(63) Continuation of application No. PCT/JP2023/018458, filed on May 17, 2023.

(30) Foreign Application Priority Data
Sep. 21, 2022 (JP) 2022-150400

(51) Int. Cl. A61B 5/00; H04W 4/80; H04W 12/03

(52) U.S. Cl. CPC A61B 5/0015; A61B 5/0004; H04W 4/80; H04W 12/03

(57) ABSTRACT

A measurement device, an information terminal, and an operation recording medium of the information terminal that can easily and safely perform pairing. A processor of a blood pressure monitor performs first control of transmitting first information including a first time from a communication unit when a short press of a measurement start button is detected, performs second control of displaying the first time on a display unit and decreasing the first time, and shifts to a pairing mode that can execute processing of sharing, with an information terminal, encryption information for performing encrypted communication by near-field wireless communication when a short press of the measurement start button is detected during a period from when the first time is displayed on the display unit to when the time displayed on the display unit reaches a predetermined value.

### CROSS-REFERENCE TO RELATED APPLICATIONS

This application is a continuation under 35 U.S.C. §120 of International Application No. PCT/JP2023/018458, filed May 17, 2023, which claims priority to Japanese Application No. 2022-150400, filed Sep. 21, 2022, under 35 U.S.C. §119(a). Each of the above-referenced patent applications are incorporated by reference in their entireties.

### TECHNICAL FIELD

The present invention relates to a measurement device, an information terminal, and an operation recording medium for an information terminal.

### BACKGROUND

The measurement device described in the present specification includes a biological information measurement device that measures biological information such as a weight, a body composition, a blood pressure, a pulse, a heart rate, a body temperature, blood glucose, or a blood oxygen saturation level, and an activity amount measurement device that measures an activity amount such as the number of steps, a walking distance, or calorie consumption. The measurement device includes a measurement sensor for measuring a measurement target amount. The measurement target amount of the measurement sensor includes biological information such as a weight, a body fat percentage, a blood pressure value, a pulse rate, a heart rate, a body temperature, a blood glucose level, or a blood oxygen saturation level, and an activity amount such as the number of steps, a walking distance, or calorie consumption, depending on the measurement device. A measurement result of such a measurement device is recorded and analyzed by an information terminal such as a smartphone, a tablet terminal, a notebook personal computer, and a desktop personal computer.

In the case of recording and analyzing such a measurement result, it is desirable that the measurement device and the information terminal are communicably connected to each other so that the information terminal can automatically acquire the measurement result, instead of the user inputting the measurement result to the information terminal each time. Specifically, for example, a method in which the information terminal receives the measurement result from the measurement device by near-field wireless communication such as Bluetooth (trade name) is conceivable.

Patent Literature 1 describes a sensor attached to a patient, the sensor including an information acquisition unit that acquires biological information of the patient; a wireless communication unit that transmits an advertising signal including sensor identification information for identifying the sensor; and a control unit that causes the wireless communication unit to transmit a confirmation signal for establishing communication authentication to a portable information terminal when the wireless communication unit receives a request for the communication authentication from the portable information terminal and causes the wireless communication unit to transmit a sensor signal corresponding to the biological information after the communication authentication is established.

Patent Literature 2 discloses a communication device including a communication means that communicates with an external device; a first reading means that reads predetermined information for specifying a type of the external device from a captured image of the external device; a presentation means that presents an operation procedure for setting connection information corresponding to the type of the external device specified from the predetermined information; a second reading means that reads the connection information from the captured image of the external device based on a display form of the connection information corresponding to the external device; and a control means that connects to the external device by the communication means using the connection information obtained by the second reading means.

Patent Literature 3 describes a communication terminal registration authentication system including a plurality of communication terminals that transmit and receive application information, a management server that manages and authenticates the plurality of communication terminals, and a network that communicably connects the plurality of communication terminals and the management server. Registration authentication of a second communication terminal is performed in response to a request from a first communication terminal.

Patent Literature 4 describes a wireless communication terminal including a first communication module that performs wireless communication with an external terminal using a first communication protocol; a second communication module that performs wireless communication with an external terminal using a second communication protocol; and a generation unit that, when the first communication module receives a connection request for requesting a connection and terminal identification information for identifying the external terminal from the external terminal according to the first communication protocol, generates a network identifier for identifying a network according to the second communication protocol based on the terminal identification information, and causes the second communication module to establish a logical link of communication with the external terminal using the network identifier and then activate the network based on the network identifier to a state in which communication with the external terminal is possible by a layer higher than a layer in which the logical link of communication is established.

### CITATION LIST

### Patent Literature

Patent Literature 1: JP 2019-115377 A

Patent Literature 2: JP 2020-120295 A

Patent Literature 3: JP 2019-32646 A

Patent Literature 4: WO 2016/189613

### SUMMARY

In order to perform secure near-field wireless communication between a measurement device and an information terminal, processing of pairing the measurement device and the information terminal is necessary. The pairing refers to processing of sharing encryption information used for near-field wireless communication between the measurement device and the information terminal. When a state in which the measurement device can be paired with the information terminal is maintained for a long period of time, there is a possibility that the measurement device is paired with an unintended information terminal, and there is a concern about safety.

An object of the present invention is to provide a measurement device, an information terminal, and an operation recording medium of an information terminal that can easily and safely perform pairing.

The above problems can be solved by the following configurations. Note that corresponding components and the like in the following embodiments are illustrated in parentheses, but the embodiments are not limited thereto.

(1)

A measurement device (blood pressure monitor 10) including a near-field wireless communication unit (communication unit 12), a display unit (display unit 15), and a processor (processor 11), wherein the processor performs, when a first operation (short press of a measurement start button 14A) is detected, first control (step S5) of transmitting first information including a first time (20 seconds or 0 seconds) from the near-field wireless communication unit; performs second control (step S6) of displaying the first time on the display unit and changing the first time displayed on the display unit over time; and shifts, when a second operation (short press of the measurement start button 14A) is detected during a period from when the first time is displayed on the display unit to when the time displayed on the display unit reaches a predetermined value (0 seconds or 20 seconds), to a pairing mode that can execute processing of sharing, with an information terminal, encryption information for performing encrypted communication by near-field wireless communication.

According to the configuration of (1), since the user can shift the measurement device to the pairing mode only by performing the first operation and the second operation, it is possible to easily share the encryption information with the information terminal. In addition, since it is possible to shift to the pairing mode by performing the second operation until the time displayed on the display unit reaches the predetermined value, the measurement device is not always in the pairing mode, and it is possible to prevent the encryption information from being shared with an unintended information terminal and to improve safety. In addition, since the mode is not shifted to the pairing mode only by the first operation, it is possible to prevent the mode from being shifted to the pairing mode by an erroneous operation.

(2)

The measurement device according to (1), further including a measurement sensor, wherein the first operation is an operation for starting an operation of the measurement sensor, and the processor performs the first control and the second control when measurement of a measurement target amount (blood pressure information) by the measurement sensor activated by the first operation is impossible.

According to the configuration of (2), since the first control and the second control are not performed in a case where the measurement result is normally obtained by performing the first operation, it is possible to appropriately perform the first control and the second control and to achieve power saving or the like. In addition, since the operation for shifting to the pairing mode can be the same as the operation performed at the time of normal measurement, it is possible to shift to the pairing mode without confusion.

(3)

The measurement device according to (1) or (2), wherein the second operation is identical to the first operation.

According to the configuration of (3), it is possible to shift to the pairing mode only by performing the same operation twice, and it is possible to more easily share the encryption information with the information terminal. By setting the first operation and the second operation as operations essential to the measurement device, it is possible to easily change the existing measurement device to perform the first control and the second control, and it is possible to easily improve the function of the existing measurement device by firmware update or the like.

(4)

The measurement device according to any one of (1) to (3), wherein the processor does not execute the first control and the second control when the processing is executed and the first operation is detected.

(5)

The measurement device according to any one of (1) to (4), wherein the processor broadcasts the first information.

According to the configuration of (5), it is possible to transmit the first information to the information terminal even in a state where communication with the information terminal is not established, and for example, it is possible to cause the information terminal to display the first time included in the first information. Since the user can recognize the pair for sharing the encryption information based on the relationship between the time displayed on the measurement device and the time displayed on the information terminal, it is possible to prevent establishment of communication connection with an unintended information terminal or sharing of the encryption information.

(6)

An information terminal (smartphone 20) including a near-field wireless communication unit (communication unit 22), a display unit (display unit 25), and a processor (processor 21), wherein the processor performs third control (step S11) of causing the display unit to display information instructing that a first operation (short press of a measurement start button 14A) be performed on a measurement device (blood pressure monitor 10); performs, in a state in which the third control is performed, fourth control (step S12) of acquiring first information including a first time (20 seconds or 0 seconds) transmitted from the measurement device, causing the display unit to display the first time, changing the first time displayed on the display unit over time, and causing the display unit to display information instructing that a second operation (short press of the measurement start button 14A) be performed on the measurement device; and performs processing (step S31) of sharing, with a measurement device that has transmitted a broadcast signal during a period from when the first time is displayed on the display unit to when the time displayed on the display unit reaches a predetermined value (0 seconds or 20 seconds), encryption information for performing encrypted communication by near-field wireless communication.

According to the configuration of (6), the user can share the encryption information between the measurement device and the information terminal only by performing the first operation and the second operation on the measurement device in accordance with the information displayed on the display unit. In addition, since the encryption information is shared with the measurement device that has transmitted the broadcast signal until the time displayed on the display unit reaches the predetermined value, it is possible to prevent the encryption information from being shared with an unintended measurement device and to improve safety.

(7)

The information terminal according to (6), wherein the first operation is an operation for starting an operation of a measurement sensor of the measurement device.

According to the configuration of (7), it is possible to share the encryption information with the measurement device by performing an operation performed at the time of normal measurement on the measurement device in accordance with the information displayed on the display unit. Therefore, the encryption information can be easily shared between the measurement device and the information terminal.

(8)

The information terminal according to (6) or (7), wherein the second operation is identical to the first operation.

According to the configuration of (8), it is possible to share the encryption

information with the measurement device by performing the operation performed at the time of normal measurement twice on the measurement device in accordance with the information displayed on the display unit. Therefore, the encryption information can be easily shared between the measurement device and the information terminal.

(9)

An operation recording medium for an information terminal (smartphone 20) including a near-field wireless communication unit (communication unit 22), a display unit (display unit 25), and a processor (processor 21), the operation recording medium causing the processor (processor 21) to execute performing third control of causing the display unit to display information instructing that a first operation be performed on a measurement device; performing fourth control of acquiring first information including a first time transmitted from the measurement device after performing the third control, causing the display unit to display the first time, changing the first time displayed on the display unit over time, and causing the display unit to display information instructing that a second operation be performed on the measurement device; and performing processing of sharing, with a measurement device that has transmitted a broadcast signal during a period from when the first time is displayed on the display unit to when the time displayed on the display unit reaches a predetermined value, encryption information for performing encrypted communication by near-field wireless communication.

According to the present invention, pairing between an information terminal and a measurement device can be performed easily and safely.

### BRIEF DESCRIPTION OF THE DRAWINGS

FIG. 1 is a schematic diagram illustrating a schematic configuration of a management system 100.

FIG. 2 is a sequence chart illustrating a procedure of processing when a blood pressure monitor 10 and a smartphone 20 are paired.

### DETAILED DESCRIPTION

### Overview of Management System Including Measurement Device and Information Terminal of Present Embodiment

The processor of the information terminal performs third control of displaying information instructing that the first operation be performed on the measurement device. When the user performs the first operation on the measurement device according to the information displayed by the third control, the processor of the measurement device performs the first control of transmitting the first information including the first time, and performs the second control of displaying the first time and changing the first time over time. When the processor of the information terminal acquires the first information transmitted from the measurement device, the processor performs fourth control of displaying the first time included in the first information, changing the first time over time, and displaying information instructing that the second operation be performed on the measurement device. When the user performs the second operation on the measurement device in accordance with the information displayed by the fourth control, the processor of the measurement device shifts to the pairing mode in which the processing of sharing, with the information terminal, the encryption information for performing the encrypted communication can be executed. When shifting to the pairing mode, the measurement device transmits a broadcast signal, and the information terminal that has received the broadcast signal establishes communication connection with the measurement device and shares encryption information with the measurement device.

According to the above-described method, pairing can be easily and safely performed between the measurement device and the information terminal.

Hereinafter, a configuration example of a management system 100 including a measurement device and an information terminal of the present embodiment will be described.

### System Configuration

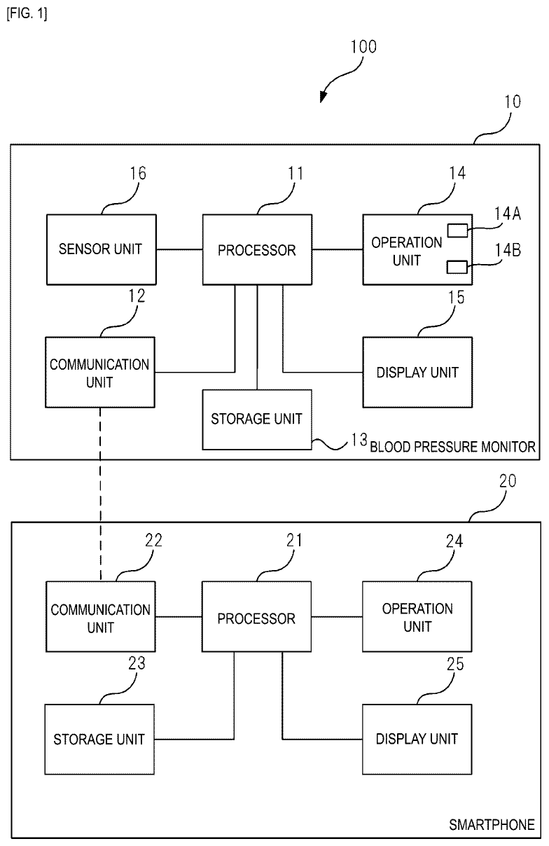

FIG. 1 is a schematic diagram illustrating a schematic configuration of the management system 100. The management system 100 includes a blood pressure monitor 10, which is an example of the measurement device, and a smartphone 20, which is an example of the information terminal, and is a system for managing measurement data, a recording medium, and the like of the blood pressure monitor 10 using the smartphone 20. The blood pressure monitor 10 and the smartphone 20 are configured to be able to communicate with each other by the near-field wireless communication. A method of the near-field wireless communication is not particularly limited, and for example, a method such as Wi-Fi, ANT, Bluetooth (trade name), or infrared communication can be used. In the following description, it is assumed that the near-field wireless communication method is Bluetooth (trade name). Hereinafter, an owner who owns both the blood pressure monitor 10 and the smartphone 20 is referred to as a user.

### Measurement Device

The blood pressure monitor 10 includes a processor 11, a communication unit 12, a storage unit 13, an operation unit 14, a display unit 15, and a sensor unit 16.

The sensor unit 16 includes a pressure sensor disposed in a cuff portion of the blood pressure monitor 10 as a measurement sensor, and detects, by the pressure sensor, a pulse wave from a blood vessel of the user under an appropriate cuff pressure. The blood pressure monitor 10 can calculate the blood pressure information including the maximum blood pressure, the minimum blood pressure, and the pulse based on the pulse wave detected by the sensor unit 16.

The communication unit 12 is a communication interface for performing the near-field wireless communication, and includes a communication antenna and various circuits.

The storage unit 13 includes a non-transitory storage medium such as a flash memory in addition to a work memory such as a random access memory (RAM). Various types of information such as measured blood pressure information are stored in this storage medium.

The operation unit 14 is an input unit such as a button or a touch panel that receives an input from the user, and receives various operations such as ON/OFF of a power supply, start of measurement of blood pressure information, and selection of an item from the user. The operation unit 14 includes a measurement start button 14A for instructing to start measuring the blood pressure information, and a communication button 14B for operating the communication unit 12 (enabling the near-field wireless communication). The measurement start button 14A and the communication button 14B may be hardware buttons, or may be software buttons displayed on the display unit 15 equipped with a touch panel.

The display unit 15 includes, for example, a display such as an organic electro-luminescence (EL) display, a liquid crystal display, or the like, and displays the measured blood pressure information and the like.

The processor 11 comprehensively controls each unit of the blood pressure monitor 10. The processor 11 is, for example, a central processing unit (CPU) that is a general-purpose processor that executes software (recording medium) to perform various functions, a programmable logic device (PLD) that is a processor whose circuit configuration can be changed after manufacturing, such as a field programmable gate array (FPGA), or a dedicated electric circuit that is a processor having a circuit configuration dedicatedly designed to execute specific processing, such as an application specific integrated circuit (ASIC). The processor 11 may be configured by one processor, or may be configured by a combination of two or more processors of the same type or different types (for example, a plurality of FPGAs or a combination of a CPU and an FPGA). For example, the processor 11 may include a processor that controls communication using the communication unit 12 and a processor that performs various types of control other than the communication. More specifically, the hardware structure of the processor 11 is an electric circuit (circuitry) in which circuit elements such as semiconductor elements are combined.

When detecting pressing of the measurement start button 14A included in the operation unit 14, the processor 11 receives an instruction to start measuring, supplies air to the cuff, and calculates the blood pressure information based on the pulse waves detected by the sensor unit 16 under an appropriate pressure of the cuff. Then, the processor 11 displays the calculated blood pressure information on the display unit 15. The processor 11 controls each component of the blood pressure monitor 10 so as to execute processing according to a user's operation performed via the operation unit 14. The action of pressing the measurement start button 14A constitutes an operation of starting the operation of the sensor unit 16. The measurement start button 14A can input a measurement start instruction to the processor 11 only by pressing the measurement start button 14A once in a short time (performing a so-called short press operation) instead of continuously pressing the measurement start button 14A for a predetermined time (performing a so-called long press operation).

In detecting the pressing of the communication button 14B included in the operation unit 14, the processor 11 activates the communication unit 12. The pressing operation of the communication button 14B necessary for activating the communication unit 12 is, for example, a long press operation.

In FIG. 1, the blood pressure monitor 10 is illustrated as an example of the measurement device, but the blood pressure monitor 10 can be replaced with a weight scale, a body composition meter, a pulse rate meter, a heart rate meter, a thermometer, a blood glucose meter, a pulse oximeter, an activity amount meter, or the like. In any of these measurement devices, the sensor unit 16 includes various measurement sensors (a pressure sensor, a pulse wave sensor, a blood glucose sensor, a photoelectric sensor, a temperature sensor, an acceleration sensor, or the like) for measuring a physical quantity of a measurement target. In a case where the measurement device is a biological information measurement device, when the processor 11 detects pressing of a measurement start button 14A included in the operation unit 14, the processor 11 operates a measurement sensor (a pressure sensor, a pulse wave sensor, a blood glucose sensor, a photoelectric sensor, a temperature sensor, or the like) included in the sensor unit 16 to measure biological information. In a case where the measurement device is an activity amount measurement device, when a power button for turning on the device is pressed instead of the measurement start button 14A, the processor 11 operates a measurement sensor (an acceleration sensor, an angular velocity sensor, or the like) included in the sensor unit 16 to measure an activity amount.

### Information Terminal

The smartphone 20 includes a processor 21, a communication unit 22, a storage unit 23, an operation unit 24, and a display unit 25.

The communication unit 22 is a communication interface for performing the near-field wireless communication, and includes a communication antenna and various circuits.

The storage unit 23 includes a non-transitory storage medium such as a flash memory in addition to a work memory such as a RAM. The storage medium stores various types of information including an application recording medium (an operation recording medium of the information terminal, a management application described later).

The operation unit 24 is an input unit such as a button or a touch panel that receives an input from a user, and receives various operations from the user.

The display unit 25 includes, for example, a display such as an organic EL display, a liquid crystal display, or the like.

The processor 21 comprehensively controls each unit of the smartphone 20. Similarly to the processor 11, the processor 21 includes one or a plurality of processors. The processor 21 executes various recording mediums stored in the storage unit 23 to perform processing according to the recording mediums.

### Method for Pairing Blood Pressure Monitor and Smartphone

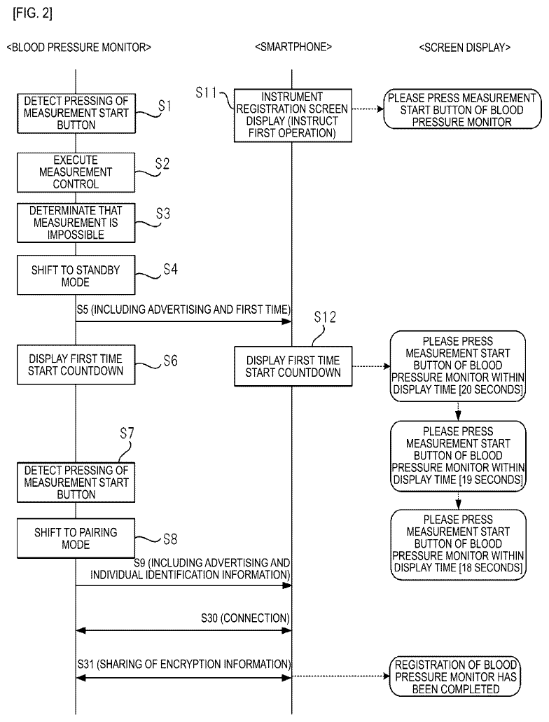

Next, a method for pairing the blood pressure monitor 10 and the smartphone 20 will be described. FIG. 2 is a sequence chart illustrating a procedure of processing when pairing between the blood pressure monitor 10 and the smartphone 20 is performed. Examples of messages each displayed on the display unit 25 of the smartphone 20 are illustrated at the right end of FIG. 2. It is assumed that a management application for managing the blood pressure information is installed and stored in advance in the storage unit 23 of the smartphone 20.

The user operates the smartphone 20 to activate the management application. When the management application is activated, the processor 21 of the smartphone 20 causes the display unit 25 to display an instrument registration screen (step S11). The processor 21 causes the communication unit 22 to start scanning. Scan means that the communication unit 22 enters a reception state and acquires information of surrounding devices. In step S11, the processor 21 of the smartphone 20 causes the display unit 25 to display the instrument registration screen including information for instructing that the first operation be performed on the blood pressure monitor 10. The first operation is, for example, an operation of starting the operation of the sensor unit 16 included in the blood pressure monitor 10, and to be specific, is “pressing the measurement start button 14A”.

As illustrated in FIG. 2, the instrument registration screen displayed in step S11 includes a message such as “Please press the measurement start button of the blood pressure monitor”. When the user confirms this message, the user prepares the blood pressure monitor 10 to be paired with the smartphone 20, turns on the power of the blood pressure monitor 10, and then presses the measurement start button 14A included in the operation unit 14 of the blood pressure monitor 10. Here, the user presses the measurement start button 14A in a state in which the cuff is not wound around the arm or the wrist, instead of performing a normal operation at the time of measuring the blood pressure information such as pressing the measurement start button 14A after winding the cuff around the arm or the wrist.

When the processor 11 of the blood pressure monitor 10 detects pressing of the measurement start button 14A (step S1), the processor 11 executes measurement control of blood pressure information (step S2). The measurement control includes control of supply and discharge of air to and from the cuff, detection of the cuff pressure by the pressure sensor, and processing of deriving blood pressure information based on pressure information detected by the pressure sensor. When the measurement start button 14A is pressed in a state in which the cuff is not wrapped around the arm or the wrist, the output of the pressure sensor becomes a value far away from that in a state in which the cuff is wrapped around the arm or the wrist. Therefore, the processor 21 of the smartphone 20 determines that the blood pressure information cannot be measured (step S3) and causes the display unit 25 to display information indicating a measurement error when the output of the pressure sensor indicates an abnormal value during execution of the measurement control.

When it is determined in step S3 that the blood pressure information cannot be measured, the processor 11 of the blood pressure monitor 10 shifts to a standby mode in which shift to the pairing mode is waited (step S4). The pairing mode is a mode in which processing of sharing, with the information terminal, encryption information for performing encrypted communication by near-field wireless communication can be executed. When it is determined that the blood pressure information can be measured, the processor 11 of the blood pressure monitor 10 stores the derived blood pressure information in the storage unit 13, displays the blood pressure information on the display unit 15, and ends the processing. That is, the processor 11 of the blood pressure monitor 10 shifts to the standby mode only when it is determined that the blood pressure information cannot be measured after the pressing of the measurement start button 14A is detected.

In the standby mode, the processor 11 of the blood pressure monitor 10 activates the communication unit 12 and causes the communication unit 12 to start advertising (step S5). The advertising refers to broadcasting a packet including various kinds of information. Hereinafter, a signal transmitted by the advertising is also referred to as a broadcast signal. In step S5, a packet including first information including mode information indicating that the device is operating in the standby mode and a first time that is a time during which the standby mode is continued is periodically broadcast. The processor 11 of the blood pressure monitor 10 causes the display unit 15 to display the first time and the mode information, and performs control (step S6) of decreasing the first time displayed on the display unit 15 over time. The first time is a freely determined time, and is 20 seconds as an example. Therefore, in step S6, an image indicating that the mode is the standby mode and the characters “20 seconds” are displayed on the display unit 15 of the blood pressure monitor 10, and every time one second elapses, the characters of the number of seconds being displayed are decremented by one second to update the time display, and the time is counted down.

When the processor 21 of the smartphone 20 acquires the first information broadcast from the blood pressure monitor 10 by the processing of step S5 via the communication unit 22, the processor 21 performs control (step S12) of displaying the first time and the mode information included in the first information on the display unit 25, reducing the first time displayed on the display unit 25 over time, and displaying information instructing that the second operation be performed on the blood pressure monitor 10 on the display unit 25. At this time, when the first information is broadcast from a plurality of instruments, the processor 21 of the smartphone 20 preferably selects an instrument having the maximum radio wave strength of the broadcast signal, acquires the first information from the selected instrument, and causes the display unit 25 to display the first time and the mode information. The second operation is preferably the same operation as the first operation (pressing of the measurement start button 14A), but may be an operation different from the first operation (for example, the short press of the communication button 14B).

As a result of the processing in step S12, a message such as “Please press the measurement start button of the blood pressure monitor within the display time. [20 seconds]” is displayed on the display unit 25 as illustrated in FIG. 2, and every time one second elapses, the characters of the number of seconds being displayed are decremented by one second to update the time display, and the time is counted down.

The processing of step S6 in the blood pressure monitor 10 and the processing of step S12 in the smartphone 20 are performed with almost no time difference. Therefore, the time displayed on the display unit 15 of the blood pressure monitor 10 and the time displayed on the display unit 25 of the smartphone 20 are updated and decreased at the same timing from “20 seconds” as an initial value. By synchronizing the time displayed on the display unit 15 of the blood pressure monitor 10 and the time displayed on the display unit 25 of the smartphone 20, the user can clearly recognize the blood pressure monitor 10 to be operated.

When the user confirms the message displayed on the smartphone 20 in the processing of step S12, the user performs the second operation (here, pressing of the measurement start button 14A) on the blood pressure monitor 10 until the number of seconds displayed reaches 0 seconds. When the processor 11 of the blood pressure monitor 10 operating in the standby mode detects pressing of the measurement start button 14A (step S7), the processor 11 shifts to the pairing mode without executing measurement control of the blood pressure information (step S8). In shifting to the pairing mode, the processor 11 of the blood pressure monitor 10 causes the communication unit 12 to start advertising (step S9). In step S9, a packet including information (for example, individual identification information of blood pressure monitor 10 or the like) necessary for establishing communication connection by near-field wireless communication is periodically broadcast.

When the processor 21 of the smartphone 20 acquires the broadcast signal transmitted from the blood pressure monitor 10 in the processing of step S9, the processor 21 issues a connection request to the blood pressure monitor 10 via the communication unit 22. When the processor 11 of the blood pressure monitor 10 responds to the connection request via the communication unit 12, a communication connection by the near-field wireless communication is established between the blood pressure monitor 10 and the smartphone 20 (step S30).

A method by which the smartphone 20 specifies a partner to which a connection request is issued is not particularly limited. For example, the processor 21 of the smartphone 20 specifies a device having the maximum radio wave strength of the broadcast signal as the connection partner, or displays a list of connection destination candidates and specifies a device selected by the user from the list as the connection partner.

When the communication connection between the smartphone 20 and the blood pressure monitor 10 is established, mutual authentication between the smartphone 20 and the blood pressure monitor 10 is performed by a predetermined method, and when the mutual authentication is completed, encryption information (for example, an encryption key or the like) for performing encrypted communication by near-field wireless communication is shared between the smartphone 20 and the blood pressure monitor 10 (step S31). The sharing of the encryption information means that the processor 11 of the blood pressure monitor 10 generates the encryption information, stores the encryption information in the storage unit 13, and transmits the encryption information to the smartphone 20, and the smartphone 20 stores the encryption information in the storage unit 23. The processor 21 of the smartphone 20 may generate the encryption information, store the encryption information in the storage unit 23, and transmit the encryption information to the blood pressure monitor 10, and the blood pressure monitor 10 may store the encryption information in the storage unit 13 to share the encryption information.

After step S31, the processor 21 of the smartphone 20 causes the display unit 25 to display, for example, a message indicating that pairing has been completed, such as “Registration of the blood pressure monitor has been completed”. Thereafter, the processor 21 of the smartphone 20 disconnects the communication connection with the blood pressure monitor 10. Thereafter, secure communication using the encryption information becomes possible between the smartphone 20 and the blood pressure monitor 10. For example, when the measurement start button 14A is pressed in a state in which the cuff is wrapped around the arm or the wrist, the blood pressure information is derived and stored in the storage unit 13. Then, the processor 11 of the blood pressure monitor 10 activates the communication unit 12, establishes a connection with the smartphone 20 with which the encryption information has been shared, encrypts the blood pressure information, and transmits the encrypted blood pressure information to the smartphone 20.

As described above, according to the management system 100, the user can shift the blood pressure monitor 10 to the pairing mode only by sequentially performing the first operation and the second operation on the blood pressure monitor 10 in accordance with the information displayed on the smartphone 20. After the shift to the pairing mode, the encryption information is shared between the blood pressure monitor 10 and the smartphone 20 by the same processing as that described above. In comparison with a configuration in which the blood pressure monitor 10 can be shifted to the pairing mode by a long press operation of a specific button, an operation of a button provided at a location where it is difficult to operate such as a bottom surface of an instrument, or the like, the management system 100 can shift the blood pressure monitor 10 to the pairing mode by the first operation and the second operation which are simple operations. Therefore, pairing of the blood pressure monitor 10 and the smartphone 20 can be easily performed. In addition, since it is possible to shift to the pairing mode by performing the second operation until the time displayed on the display unit 15 and the display unit 25 reaches the predetermined value (“0 seconds” in the above example) (that is, the duration of the standby mode), the blood pressure monitor 10 is not always in the pairing mode, and it is possible to prevent the encryption information from being shared with an unintended information terminal and to improve safety.

In addition, since the processor 11 of the blood pressure monitor 10 does not shift to the pairing mode only by the first operation being performed, it is possible to prevent the processor 11 from shifting to the pairing mode due to an erroneous operation. The processor 11 of the blood pressure monitor 10 does not unconditionally shift to the standby mode when the first operation is performed, but shifts to the standby mode when it is determined that the blood pressure information cannot be measured. Therefore, at the time of normal blood pressure measurement, it is possible to operate the processor 11 in the standby mode and the pairing mode only when necessary, without shifting to the standby mode and the pairing mode. The processor 11 of the blood pressure monitor 10 may not shift to the standby mode even when the first operation is performed in a case where the processing of sharing the encryption information has been executed. That is, in a case where the processing of sharing the encryption information has been executed and the first operation is detected (in a case where the first operation is detected after the processing of step S31 in FIG. 2), the processor 11 may start the blood pressure measurement processing without executing the control of shifting to the standby mode and the pairing mode. In this way, it is possible to prevent shift to the standby mode and the pairing mode in a case where the first operation is erroneously performed, and to achieve power saving or the like.

In the management system 100, by making the first operation and the second operation the same, it is possible to easily and safely perform pairing even when there is a limit to the number of operators (buttons or the like) included in the operation unit 14 mounted on the blood pressure monitor 10. In particular, by setting the first operation and the second operation as operations essential to the blood pressure monitor 10 (operations for starting measurement of blood pressure information), it is possible to cause many existing blood pressure monitors 10 to execute the processing illustrated in FIG. 2, and it is possible to reduce the construction cost of the management system 100 by eliminating the need to modify the hardware of the blood pressure monitor 10.

In the above description, the first operation is pressing of the measurement start button 14A, but pressing (short press) of the communication button 14B may be used as the first operation, or pressing of a power button (a button for turning on the power of the blood pressure monitor 10) (not illustrated) may be used as the first operation. That is, in step S11, the operation of the communication button 14B (or the power button) may be instructed instead of the measurement start button 14A, and when the pressing of the communication button 14B (or the power button) is detected in step S1, the processing from step S4 to step S6 may be performed. In this case, the processing of step S2 and the processing of step S3 are omitted, and the operation is changed to an operation of displaying information for instructing the operation of the communication button 14B (or the measurement start button 14A) on the display unit 25 in step S12 and shifting to the pairing mode when the pressing of the communication button 14B (or the measurement start button 14A) is detected in step S7.

In addition, in the above description, the first time is displayed and then the first time is decreased in step S6 and step S12. However, the first time may be displayed and then the first time may be increased. For example, the first time may be set to “0 seconds”, and in step S6 and step S12, control may be performed to increase the displayed number of seconds by “1” every time one second elapses. In this case, for example, a message such as “Please press the measurement start button of the blood pressure monitor 10 until the displayed number of seconds reaches 20 seconds. [0 seconds]” will be displayed on the display unit 15 and the display unit 25.

Although various embodiments have been described above, it is needless to say that the present invention is not limited to such examples. It will be apparent to those skilled in the art that various changes and modifications can be made within the scope of the claims, and it is understood that these are naturally belong within the technical scope of the present invention. Further, components of the above-described embodiments may be combined as desired within a range that does not depart from the spirit of the present invention.

This application is based on a Japanese patent application filed on Sep. 21, 2022 (JP 2022/150400), the contents of which are incorporated herein by reference.

### REFERENCE NUMERALS LIST

10 Blood pressure monitor

### 11, 21 Processor

12, 22 Communication unit
13, 23 Storage unit
14A Measurement start button
14B Communication button
14, 24 Operation unit
15, 25 Display unit
16 Sensor unit

### 20 Smartphone

100 Management system

What is claimed is:

1. A measurement device comprising:
a near-field wireless communication unit;
a display unit; and
a processor, wherein
the processor
performs, when a first operation is detected, first control of transmitting first information including a first time from the near-field wireless communication unit;
performs second control of displaying the first time on the display unit and changing the first time displayed on the display unit over time; and
shifts, when a second operation is detected during a period from when the first time is displayed on the display unit to when the time displayed on the display unit reaches a predetermined value, to a pairing mode configured to execute processing of sharing, with an information terminal, encryption information for performing encrypted communication by near-field wireless communication.

2. The measurement device according to claim 1, further comprising:
a measurement sensor, wherein
the first operation is an operation for starting an operation of the measurement sensor, and
the processor performs the first control and the second control when measurement of a measurement target amount by the measurement sensor activated by the first operation is impossible.

3. The measurement device according to claim 2, wherein the second operation is identical to the first operation.

4. The measurement device according to claim 1, wherein the processor does not execute the first control and the second control when the processing is executed and the first operation is detected.

5. The measurement device according to claim 1, wherein the processor broadcasts the first information.

6. An information terminal comprising:
a near-field wireless communication unit;
a display unit; and
a processor, wherein
the processor
performs third control of causing the display unit to display information instructing that a first operation be performed on a measurement device;
performs, in a state in which the third control is performed, fourth control of acquiring first information including a first time transmitted from the measurement device, causing the display unit to display the first time, changing the first time displayed on the display unit over time, and causing the display unit to display information instructing that a second operation be performed on the measurement device; and
performs processing of sharing, with a measurement device that has transmitted a broadcast signal during a period from when the first time is displayed on the display unit to when the time displayed on the display unit reaches a predetermined value, encryption information for performing encrypted communication by near-field wireless communication.

7. The information terminal according to claim 6, wherein the first operation is an operation for starting an operation of a measurement sensor of the measurement device.

8. The information terminal according to claim 6, wherein the second operation is identical to the first operation.

9. An operation recording medium for an information terminal including a near-field wireless communication unit, a display unit, and a processor, the operation recording medium causing the processor to execute:
performing third control of causing the display unit to display information instructing that a first operation be performed on a measurement device;
performing fourth control of acquiring first information including a first time transmitted from the measurement device after performing the third control, causing the display unit to display the first time, changing the first time displayed on the display unit over time, and causing the display unit to display information instructing that a second operation be performed on the measurement device; and
performing processing of sharing, with a measurement device that has transmitted a broadcast signal during a period from when the first time is displayed on the display unit to when the time displayed on the display unit reaches a predetermined value, encryption information for performing encrypted communication by near-field wireless communication.
